(19) United States
(12) Patent Application Publication
(10) Pub. No.: US 2004/0230088 A1
(43) Pub. Date: Nov. 18, 2004

(54) Flexoelectric apparatus

(76) Inventor: Paul Yanick JR., Kingston, NY (US)

Correspondence Address: JOHN MAIER, III, 666 AARON COURT, KINGSTON, NY, 12401, US

(21) Appl. No.: 10/833,643

(22) Filed: Apr. 28, 2004

Related U.S. Application Data
(60) Provisional application No. 60/465,735, filed on Apr. 28, 2003.

(51) Int. Cl. A61N 1/00

(52) U.S. Cl. 600/13

(57) ABSTRACT

Two versions of a flexoelectric apparatus are taught. Both produce electromagnetic fields to aid in the curing of illness in the body. One version, known as a Quantum Coherence Generator, is a flexoelectric harmonic oscillator coil that is driven by natural life sustaining frequencies. The apparatus includes a coherence coil, which is a harmonic oscillator and which has four and half turns, the coil being made from copper wire in the range of sixteen to thirty gauge.

The other version, known as the Quantum Energizer, by which an electromagnetic field of cell is augmented. It includes an enclosure of a flexoelectric mineral matrix or silica with an energizer coil within the enclosure made from a copper wire and having three and one-half turns with a triangle made of gold at the outside perimeter of the energizer coil.

### RELATED APPLICATIONS

The portion of this application which pertains to a Quantum Coherence Generator was previously filed as a Provisional Application on Apr. 28, 2003 as Application Serial No. 60/465,735 and priority based upon that Provisional Application is claimed.

### BACKGROUND OF THE INVENTION

1. Field of the Invention

This invention relates to flexoelectric apparatus and, most specifically, flexoelectric apparatus for treating living beings, both human and animals, by reestablishing cellular resonance and eliminating chaos and by moving coherent energy deep into an unhealthy organism.

2. Prior Art

Numerous bio-electromagnetic studies indicate that weak electromagnetic fields can have profound physiological effects on a variety of biological issues.¹ Dr. Glen Rein² performed studies to demonstrate a direct biochemical effect of quantum fields at the cellular level. Dr. Rein utilized a device known as a cadiuceius coil and cells treated with the cadiuceius coil showed, according to Dr. Rein, a twenty fold simulation of cell growth. ¹Rein: The Body Question: Non-classical Behavior of Biological Systems. ²Rein, Supra.

Biophotons are a driving force behind molecular coherence and at different frequencies biophotons emit coherent emissions near the Zero Point Field while individuals with cancer have chaotic admissions. Therefore, the health building efforts of restoring quantum coherence reasonably may have far reaching health benefits.

One version of the flexoelectric apparatus, known as the Quantum Coherence Generator, produces spiraling movements that break up cellular and sub-cellular chaos and clusters of protein made when genetic expression is altered with genes producing mutilated proteins instead of functional proteins. The rapid swirling of these coherent energies drive the potential to restore the order of biomolecular and bio-chemical networks more fully to engage the body's natural power of self healing.

Another version of the flexoelectric device is a Quantum Energizer by which the electromagnetic field of cells is augmented. This was partially achieved by Dr. Rein with his Cadiuceius Coil but the Quantum Energizer provides distinct advantages over the Cadiuceius Coil. The Quantum Energizer energizes innate healing mechanisms to perform at higher levels of efficiency and functional states. This boost in electromagnetic healing energy allows the body to move beyond its reduced energetic capacity. The Quantum Energizer is a coherence generator plus an energizer as it primarily energizes and protects from outside a chaotic environmental influence on an unlimited and dynamic time scale.

### Objects

The objects of this invention are to provide as follows:

1. A flexoelectric apparatus to promote innate healing.

2. A flexoelectric apparatus to produce coherence that is driven by natural life-sustaining frequencies.

3. A flexoelectric apparatus to boost electromagnetic healing energy.

4. A flexoelectric apparatus that is economical to produce and is effective.

### SUMMARY OF THE INVENTION

A flexoelectric apparatus is provided to produce coherence known more specifically as a Quantum Coherence Generator. Such flexoelectric apparatus includes a coherence coil formed from a copper wire. The coherence coil has a base and an outside perimeter and has four and one half turns with a distance between turns of one-quarter of an inch. Each turn toward the outside perimeter from the base having a successively smaller diameter.

The other flexoelectric apparatus which is used to energize, more specifically known as a Quantum Energizer, includes an energizer coil made from a copper wire with a base and an outside perimeter. The energizer coil has three and one-half turns between the base and the outside perimeter. Each successive turn from the base to the outside perimeter has a smaller diameter. A triangle made of gold is mounted on the outside perimeter of the energizer coil. An enclosure which has the shape of a dome or a triangle is constructed of a flexoelectric mineral matrix or from silica. The energizer coil and the triangle are located inside the enclosure.

### DESCRIPTION OF THE DRAWINGS

FIG. 1 is a top view of the Quantum Coherence Generator.

FIG. 2 is a side view of the Quantum Coherence Generator.

FIG. 3 is a pictorial view of the Quantum Coherence Generator.

FIG. 4 is a cross-sectional side elevation of the Quantum Energizer.

FIG. 5 is a top view of the coil used in the Quantum Energizer showing the triangle on the energizer coil.

FIG. 6 is a pictorial view of the Quantum Energizer.

FIG. 7 is pictorial view of the coil utilized in the Quantum Energizer with the triangle attached.

### DESCRIPTION OF THE NUMERALS

| NUMERAL | DESCRIPTION |
|---|---|
| 11 | Coherence Coil |
| 13 | Base (Coherence Coil) |
| 15 | Outside Perimeter (Coherence Coil) |
| 17 | Enclosure |
| 19 | Bottom (Enclosure) |
| 21 | Energizer Coil |
| 23 | Base (Energizer Coil) |
| 25 | Outside Perimeter (Energizer Coil) |
| 27 | Triangle |

### DETAILED DESCRIPTION OF THE PREFERRED EMBODIMENT

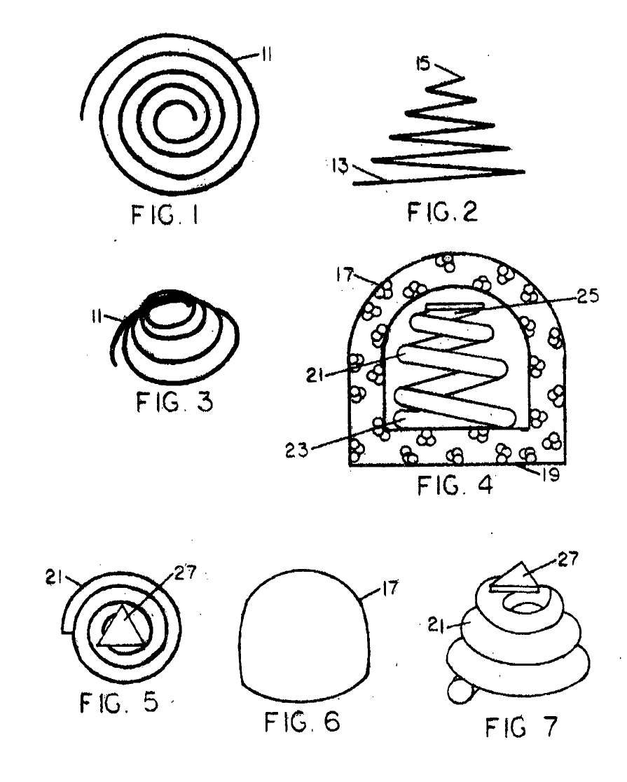

Referring now to FIGS. 1, 2 and 3, the details of the flexoelectric apparatus which produces coherence, known more specifically as a Quantum Coherence Generator, are shown. The details of the Quantum Coherence Generator are critical. The Quantum Coherence Generator is a coherence coil 11, which is harmonic oscillator coil, made from copper and which has four and a half turns. The copper wire from which the coherence coil 11 is formed has a circular cross section within the range of sixteen to thirty gauge wire. The coherence coil 11 has a base 13 and a outside perimeter 15. The coherence coil 11 at the base 13 has a diameter of four inches and the distance between ascending turns is one-quarter of an inch. The diameter of each ascending turn is less that the diameter of the turn directly beneath it toward the base 13. The distance between the base 13 and the outside perimeter 15 is two and one quarter inches.

The flexoelectric apparatus for energizing, known as the Quantum Energizer, is encased within an enclosure 17 shown in FIGS. 4 through 7. The enclosure 17, which includes a bottom 19, is constructed from a flexoelectric mineral matrix that is a combination of rare-earth minerals, gemstones and crystalline nano-structures. The enclosure may also be constructed of silica. The enclosure 17 has a thickness of substantially three-quarters of an inch. The outside diameter of the bottom 19 of the enclosure 17 is four and one half inches. The internal diameter of the bottom 19 of the enclosure 17 is three inches. An energizer coil 21 is made from a copper wire that has a circular cross section and is in the range of sixteen to thirty gauge wire. The energizer coil 21 has a base 23 and an outside perimeter 25. The base 23 of the energizer coil 21 has a diameter of two and one-half inches and has three and one-half turns with a half inch between successive turns. Each successive turn has a smaller diameter than the turn immediately beneath it toward the base 23. A triangle 27 made of gold is secured to the outside perimeter 25 of the energizer coil 21. The triangle 27 has a thickness of wire in the range of sixteen gauge to thirty gauge.

While a preferred embodiment has been shown and described, it will be apparent to those skilled in the art that many other changes and modifications may be made without departing from the invention in its broader aspects. The appended claims are therefore intended to cover all such changes and modifications as fall within the true spirit and scope of the invention.

1. A flexoelectric apparatus to produce coherence known more specifically as a Quantum Coherence Generator, the flexoelectric apparatus comprising:
a coherence coil formed from a copper wire, the coherence coil having a base and an outside perimeter and having four and one half turns with a distance between turns of one-quarter of an inch, each turn toward the outside perimeter from the base having a successively smaller diameter.

2. A flexoelectric apparatus according to claim 1 wherein the copper wire has a circular cross section and is in the range of sixteen to thirty gauge wire.

3. A flexoelectric apparatus according to claim 1 wherein the coherence coil has a diameter at its base of four inches.

4. A flexoelectric apparatus according to claim 1 wherein the distance between the base and the outside perimeter is two and one quarter inches.

5. A flexoelectric apparatus to produce coherence known more specifically as a Quantum Coherence Generator, the flexoelectric apparatus comprising:
a coherence coil formed from a copper wire with a circular cross section and being in the range of sixteen to thirty gauge wire, the coil having a base and an outside perimeter, the diameter of the coherence coil at the base being four inches and having four and one half turns with a distance between turns of one-quarter of an inch, each turn toward the outsie perimeter from the base having a successively smaller diameter, the distance between the base and the outside perimeter being two and one-quarter inches.

6. A flexoelectric apparatus to energize, more specifically known as a Quantum Energizer, said flexoelectric apparatus comprising:
an energizer coil made from a copper wire and having a base and an outside perimeter, and having three and one-half turns between the base and the outsie perimeter, each successive turn from the base to the outside perimeter having a smaller diameter;
a triangle made of gold mounted on the energizer coil at the outside perimeter of the energizer coil; and
an enclosure, the energizer coil and triangle being located inside the enclosure.

7. A flexoelectric apparatus according to claim 6 wherein the copper wire of the energizer coil is in the range of sixteen to thirty gauge wire.

8. A flexoelectric apparatus according to claim 6 wherein the base of the energizer coil has a diameter of two and one half inches.

9. A flexoelectric apparatus according to claim 6 wherein the triangle has a thickness of wire in the range of sixteen gauge to thirty gauge wire.

10. A flexoelectric apparatus according to claim 6 wherein the enclosure has a thickness of three quarters of an inch.

11. A flexoelectric apparatus according to claim 6 wherein the enclosure is constructed from a flexoelectric mineral matrix, being a combination of rare earth minerals, gemstones and crystalline nano-structures.

12. A flexoelectric apparatus according to claim 6 wherein the enclosure is constructed of silica.

13. A flexoelectric apparatus according to claim 6 wherein the enclosure has the shape of a dome.

14. A flexoelectric apparatus according to claim 6 wherein the enclosure has a triangular shape.

15. A flexoelectric apparatus to energize, more specifically known as a Quantum Energizer, said flexoelectric apparatus comprising:
an energizer coil made from a copper wire in the range of sixteen to thirty gauge wire, the energizer coil having a base and a top, the base having a diameter of two and one half inches and having three and one-half turns between the base and the outside perimeter, each successive turn from the base to the outside perimeter having a smaller diameter;
a triangle made of gold mounted on the energizer coil at the outside perimeter of the energizer coil, the triangle being constructed of wire in the sixteen to thirty gauge wire; and
an enclosure having the shape of a dome and a thickness of three quarter of an inch, the enclosure being constructed of a flexoelectricc mineral matrix being a combination of rare earth minerals, gemstones and crystalline nano-structures, the energizer coil and triangle being located inside the enclosure.

16. A flexoelectric apparatus to energize, more specifically known as a Quantum Energizer, said flexoelectric apparatus comprising:
an energizer coil made from a copper wire in the range of sixteen to thirty gauge wire, the energizer coil having a base and an outsie perimeter, the base having a diameter of two and one half inches and having three and one-half turns between the base and the outside perimeter, each successive turn from the base to the outside perimeter having a smaller diameter;
a triangle made of gold mounted on the energizer coil at the outside perimeter of the energizer coil, the triangle being constructed of wire in the range of sixteen to thirty gauge wire; and
an enclosure having a thickness of three quarter of an inch.

17. A flexoelectric apparatus according to claim 16 wherein the enclosure is constructed of silica.

18. A flexoelectric apparatus according to claim 16 wherein the enclosure has the shape of a dome.

19. A flexoelectric apparatus according to claim 16 wherein the enclosure has a triangular shape.

20. A flexoelectric apparatus according to claim 16 wherein the enclosure is constructed of a flexoelectric mineral matrix being a combination of rare earth mineral, gemstones and crystalline namo-structures.
